(19) United States
(12) Patent Application Publication
(10) Pub. No.: US 2025/0367374 A1
(43) Pub. Date: Dec. 4, 2025

(54) INTEGRATED COIL MULTI-MATERIAL EXTUSION FOR INFUSION SET CANNULA

(71) Applicant: Tandem Diabetes Care, Inc., San Diego, CA (US)

(72) Inventors: Justin Coker, San Diego, CA (US); Kennie Hsu, San Diego, CA (US); Amrith Karunaratne, San Diego, CA (US)

(21) Appl. No.: 19/220,426

(22) Filed: May 28, 2025

Related U.S. Application Data
(60) Provisional application No. 63/652,275, filed on May 28, 2024.

(51) Int. Cl. A61M 5/158; A61M 5/142

(52) U.S. Cl. CPC A61M 5/158; A61M 5/14248; A61M 2202/0007; A61M 2202/04; A61M 2205/10; A61M 2207/00

(57) ABSTRACT

Disclosed herein are cannula configurations and methods for making such configurations that closely replicate the functional properties of a coil reinforced cannula within a single extrusion. By using a multi-lumen process, a tube can be manufactured with a fluid path inner diameter surrounded by a number of smaller cores embedded in the tube wall. With this process, multiple materials can be used such that the smaller cores are a harder durometer than the softer tube wall. The result is a soft flexible tube embedded with rigid helical cores that can replace stainless-steel coil reinforced extrusion.

### PRIORITY CLAIM

This application claims the benefit of U.S. Provisional Patent Application No. 63/652,275 filed May 28, 2024, which is hereby incorporated herein by reference in in its entirety.

### TECHNICAL FIELD

The present disclosure relates infusion sets that facilitate injection of medicament into a body of a patient.

### BACKGROUND

There are a wide variety of medical treatments that include the administration of a therapeutic fluid in precise, known amounts at predetermined intervals. Devices and methods that are directed to the delivery of such fluids, which may be liquids or gases, are known in the art.

One category of such fluid delivery devices includes insulin injecting pumps developed for administering insulin to patients afflicted with Type 1 or Type 2 diabetes. Some insulin injecting pumps are configured as portable or ambulatory infusion devices that can provide continuous subcutaneous insulin injection and/or infusion therapy as an alternative to multiple daily injections of insulin via a syringe or an insulin pen. Such pumps can be worn or carried by the user and may use replaceable cartridges. In some embodiments, these pumps may also deliver medicaments other than, or in addition to, insulin, such as glucagon, pramlintide, and the like. Examples of such pumps and various features associated therewith include those disclosed in U.S. Patent Publication Nos. 2013/0324928 and 2013/0053816 and U.S. Pat. Nos. 8,287,495; 8,573,027; 8,986,253; and 9,381,297, each of which is incorporated herein by reference in its entirety.

Some portable infusion pumps deliver medicament to patients through infusion sets that include tubing extending from the pump and a cannula with an associated needle positioned transcutaneously (i.e., through the patient's skin) at an infusion site to allow infusion of the medicament through a cannula and into the patient. Such pumps can be worn on the body or carried near the body (e.g., in the user's pocket) with the infusion site situated on the patient's body and connected with the pump via the tubing. Other pumps that are worn directly on the body can deliver medicament through a cannula that extends directly beneath the pump.

If a patient leaves the cannula injected at the infusion site at one location for too long a period of time, or uses the same infusion site repeatedly, unwanted side effects such as infection and the accumulation of fat and scar tissue can result. Therefore, patients are often instructed to rotate infusion sites to avoid or minimize side effects. Depending on the type of cannula used, the general physiological response of the patient with regard to insulin absorption, and other factors, the time needed between infusion site rotations can vary. Commonly, sites are rotated every 24-48 hours or every 48-72 hours and extended wear infusion sets that can be worn for a longer period of time, such as for example, 7 days, are also being developed.

Embodiments of various infusion sets are described in U.S. Patent Publication Nos. 2018/0280608, 2021/0402084, 2022/0226568 and 2023/0277765, each of which is hereby incorporated herein by reference in its entirety. Some of these infusion sets include a coil extending through the delivery cannula inserted into the user for delivery of medicament into the user's skin. Use of such a coil within the cannula can provide a number of benefits to the user including, but not limited to, flexibility and kink resistance, which increase the wear time of an infusion set. The coil can also provide a filtering function to capture and retain aggregate particles formed in the liquid medication. Such aggregate particles can induce or contribute to infusion site inflammatory and immune responses if delivered and can compromising the infusion site.

While cannulas with internal coils provide significant advantages, there are technical challenges and increased costs associated with the manufacturing process. For example, the coils must be manually inserted on a long mandrel, the coils must be manually spaced and aligned along the length of the mandrel, care must be taken so that the coils do not shift during the extrusion process, the mandrel must be removed from the extruded coils, and the extruded coils must be cut to size. These requirements, alone or in combination with one another, can be a challenge for large scale manufacturing. There are also usability challenges associated with using a cannula with metallic internal coils. For example, the cannula can become corroded over time which can impact the delivery of medicament. Additionally, the metallic internal coils are not safe for wear during MRI or CT scanning and infusion sets with metallic internal coils must be removed prior to the MRI or CT scan and discarded earlier than otherwise intended.

### SUMMARY

Disclosed herein are cannula configurations and methods for making such configurations that closely replicate the functional properties of a coil reinforced cannula within a single extrusion. By using a multi-lumen process, a tube can be manufactured with a fluid path inner diameter surrounded by a number of smaller cores embedded in the tube wall. With this process, multiple materials can be used such that the smaller cores are a harder durometer than the softer tube wall. The result is a soft flexible tube embedded with rigid helical cores that can replace stainless-steel coil reinforced extrusion.

In an embodiment, a cannula configured to facilitate delivery of insulin from an infusion pump to a user can include an elongate tube having an outer wall and an inner lumen. A helical core can be disposed within the outer wall and extend along a length of the elongate tube. The helical core can be comprised of a more rigid material than the elongate tube.

In an embodiment, a cannula configured to facilitate delivery of insulin from an infusion pump to a user can be formed with a helical core disposed within and extending along a length of an outer wall of an elongate tube. The helical core can be comprised of a more rigid material than the elongate tube.

In an embodiment, an insulin delivery system includes a cannula including an elongate tube having an outer wall and an inner lumen and a helical core disposed within the outer wall and extending along a length of the elongate tube. The helical core can be comprised of a more rigid material than the elongate tube.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIGS. 1A-1B depict an ambulatory infusion pump with an infusion set according to an embodiment of the disclosure.

FIGS. 2A-2C depict an ambulatory infusion pump according to an embodiment of the disclosure.

FIG. 3 depicts an infusion set according to an embodiment of the disclosure.

FIGS. 4A-4B depict a cannula according to an embodiment of the disclosure.

FIG. 5 depicts a flowchart of steps for a method of manufacturing a cannula according to an embodiment of the disclosure.

FIGS. 6A-6B depict a cannula formed by the method described in FIG. 5.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION

The following detailed description should be read with reference to the drawings in which similar elements in different drawings are numbered the same. The drawings, which are not necessarily to scale, depict illustrative embodiments and are not intended to limit the scope of the invention.

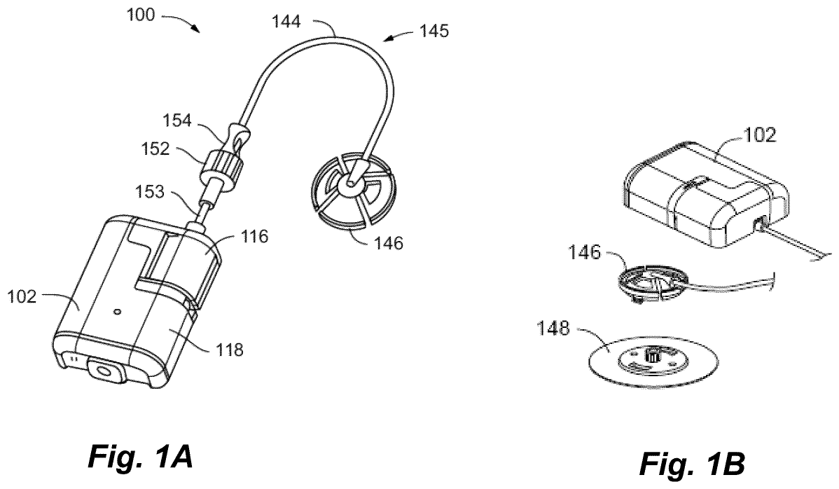

FIGS. 1A-1B depict an exemplary medical device that can be used with embodiments of the disclosure. In this embodiment, the medical device is configured as a pump 102, such as an infusion pump, that can include a pumping or delivery mechanism and a reservoir for delivering a medicament to a patient. In one embodiment, the medical device can be a portable pump configured to deliver insulin to a patient. Further details regarding such pump devices can be found in U.S. Pat. Nos. 8,287,495, 10,279,107 and 10,864,318, each of which is incorporated herein by reference in its entirety. In other embodiments, the medical device can be an infusion pump configured to deliver one or more additional or other medicaments to a patient.

As depicted in FIGS. 1A-1B, pump system 100 can include a pump 102 and an infusion set 145. In embodiments, pump system 100 can include a drive unit 118 and a cartridge 116 having a short length of tubing 153 and a connector 152 extending therefrom. Infusion set 145 can include tubing 144 extending between a connector 154 and a site connector 146. Connector 154 on infusion set 145 can be configured to couple to pump 102 at connector 152 of pump 102. As shown in FIG. 1B, site connector 146 can be configured to be attached to an infusion hub 148 at an infusion site on a user through which medicament from the pump is delivered to a patient through a cannula extending from the infusion hub 148 into the user's skin.

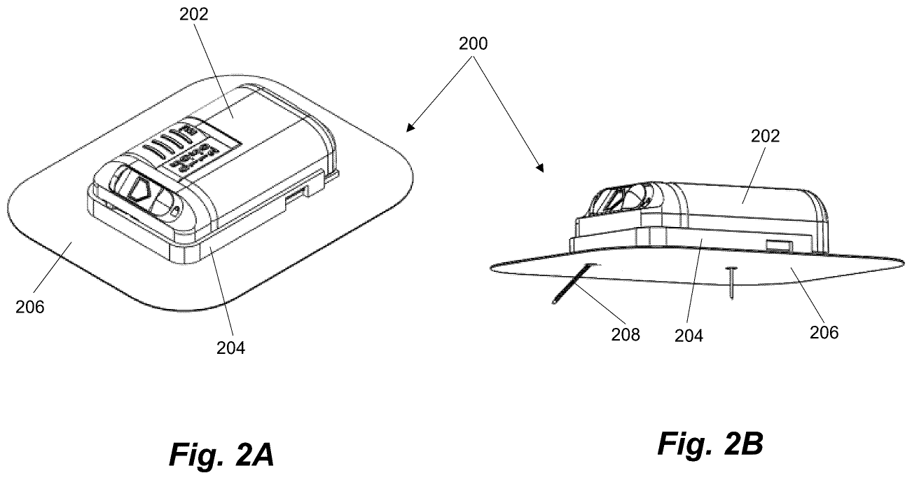

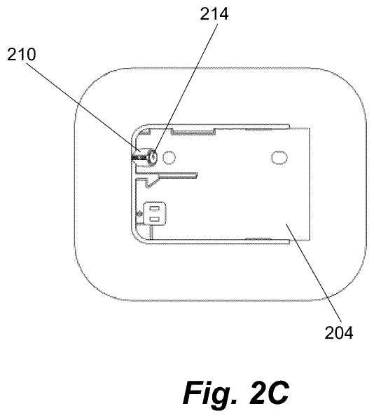

FIGS. 2A-2C depict an infusion pump system 200 according to another embodiment of the disclosure. System 200 can include an infusion pump 202 configured as a patch pump that is worn directly on the body of the user with an adhesive patch 206. Pump 202 can be affixed to the body of the user with an adhesive patch 206 carrying a tray 204 thereon that releasably attaches the pump 202 to the tray 204. Pump 202 can be configured to deliver medicament to the user through a cannula 208 extending directly beneath the pump 202 and adhesive patch 206. Referring to FIG. 2C, in embodiments tray 204 can include a cannula port 210 through which cannula 208 is inserted. The cannula port 210 can contain a septum 214 therein that facilitates transfer of insulin from the pump 202 to the cannula 208.

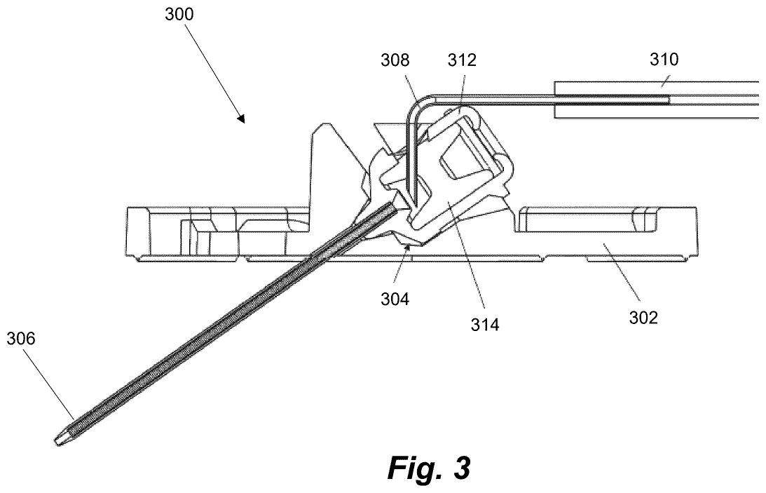

FIG. 3 depicts an infusion hub 300 of an infusion set according to an embodiment of the disclosure. Hub 300 can include a patch 302 or other mechanism configured to adhere to the patient and a barrel 304 connected to a cannula 306. A fluid introducer needle 308 can interface tubing 310 with the hub 300. The barrel 304 can include a mechanical housing 312 configured to house a septum 314. The fluid introducer needle 308 can be configured to pierce the septum 314 to deliver fluid from the tubing 310 to the cannula 306. The fluid path for the insulin medication, therefore, can run from a reservoir of a pump through infusion tubing extending from the pump, through one or more connectors connecting the infusion tubing of the pump with tubing 310 of an infusion set, through the tubing 310 of the infusion set, to the barrel 304 and to the patient via the cannula 306. Although infusion hub 300 is depicted as being employed with a length of tubing 310 that may interface with tubing extending from a pump such as depicted in FIGS. 1A-1B, the components of infusion hub 300 such as the septum 314, cannula 306, etc. can be adapted to be incorporated into a patch pump system such as the system depicted in FIGS. 2A-2B.

As noted above, providing a coil inside of a cannula such as cannula 306 has been found to provide a number of benefits. In some examples, the coil is comprised of a stainless-steel material that is co-extruded to have a polymer sleeve over its external surface. While such configurations provide the advantages of flexibility, kink resistance, and particulate filtering as noted herein, there are technical challenges and increased costs associated with the manufacturing process. For example, the coils must be manually inserted on a long mandrel, the coils must be manually spaced and aligned along the length of the mandrel, care must be taken so that the coils do not shift during the extrusion process, the mandrel must be removed from the extruded coils, and the extruded coils must be cut to size. These requirements, alone or in combination with one another, can be a challenge for large scale manufacturing. There are also usability challenges associated with using a cannula with metallic internal coils. For example, the cannula can become corroded over time which can impact the delivery of medicament. Additionally, the metallic internal coils are not safe for wear during MRI or CT scanning and infusion sets with metallic internal coils must be removed prior to the MRI or CT scan and discarded earlier than otherwise intended.

Disclosed herein are cannula configurations and methods for making such configurations that closely replicate the functional properties of a coil reinforced cannula within a single extrusion. By using a multi-lumen process, a tube can be manufactured with a fluid path inner diameter surrounded by a number of smaller cores embedded in the tube wall. With this process, multiple materials can be used such that the smaller cores are a harder durometer than the softer tube wall. It is also possible to rotate or twist the tube during the extrusion process. The result is a soft flexible tube embedded with rigid helical cores that can replace stainless-steel coil reinforced extrusion. The embedded cores can be optimized for mechanical flexibility and insulin preservative retention. The tube wall thickness, core diameter, material durometer, number of cores, etc. can all be varied as desired to provide specific properties.

Following the multi-lumen, multi-material extrusion process, the tube with embedded helical cores can be processed in a similar manner to current cannula manufacturing to provide the tube with the desired features to function as a cannula. In particular, the tube can be cut to length, distal end tipped and infusion holes laser drilled. A hub can then be molded onto the proximal end of the cannula (see, hub 304 in FIG. 3) to provide a cannula assembly for use in an infusion set such as infusion set 300. Alternatively, cannula can be configured to interface with a patch pump to deliver medicament directly beneath the pump or used with any other infusion mechanism that utilizes a cannula.

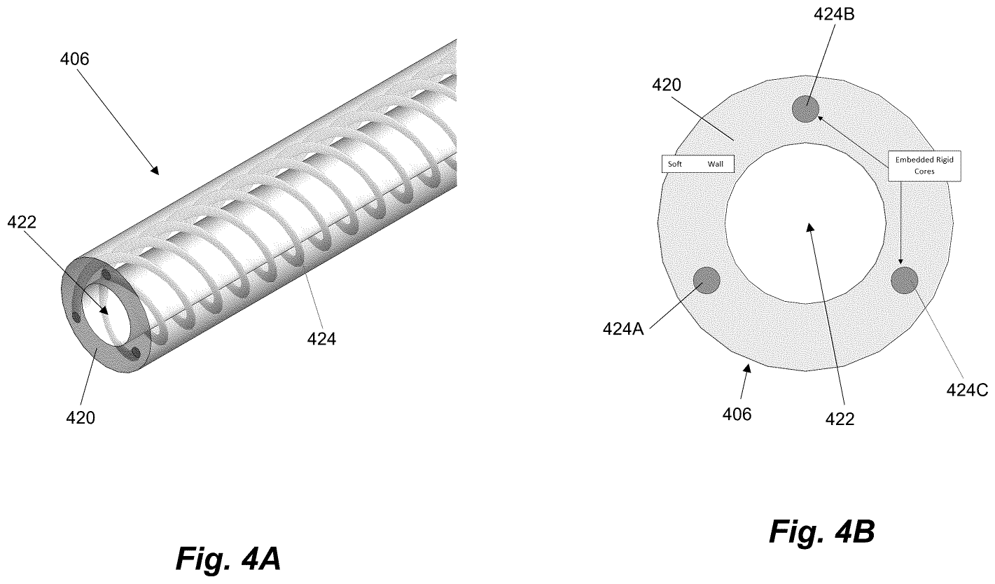

FIGS. 4A-4B depict a portion of a cannula 406 manufactured according to these processes for use in an infusion set, with a patch pump for delivery directly beneath the pump, or to otherwise deliver liquid medicament into a user. Cannula 406 includes a tube wall 420 having an inner lumen 422 through which medicament can be delivered to a user. The tube wall 420 includes an inner diameter and an outer diameter defining a wall thickness therebetween. A core 424 can be embedded within tube wall 420. Tube wall 420 may comprise a softer, more flexible polymer material, such as, for example, a thermoplastic elastomer and core 424 embedded within tube wall 420 can comprise a harder, more rigid polymer material. Core 424 may be formed of a single core material or multiple overlapping cores. Because it can be difficult to wind a single core tight enough during the extrusion process to replicate the coil spacing of a metal coil, in the depicted embodiment, three separate overlapping cores 424A, 424B, 424C are provided in order to better mimic the shape of a metal coil and therefore more closely mirror the functionality provided by a metal coil. In the depicted embodiment, the core 422 is disposed entirely within the tube wall 420 between the inner and outer diameter of the tube wall 420 such that the filtering function provided by an internal coil would not be provided. In other embodiments, core can be extruded such that a portion of the coil material protrudes inwardly into the inner lumen 422 to aid in filtering the liquid medication that flows therethrough.

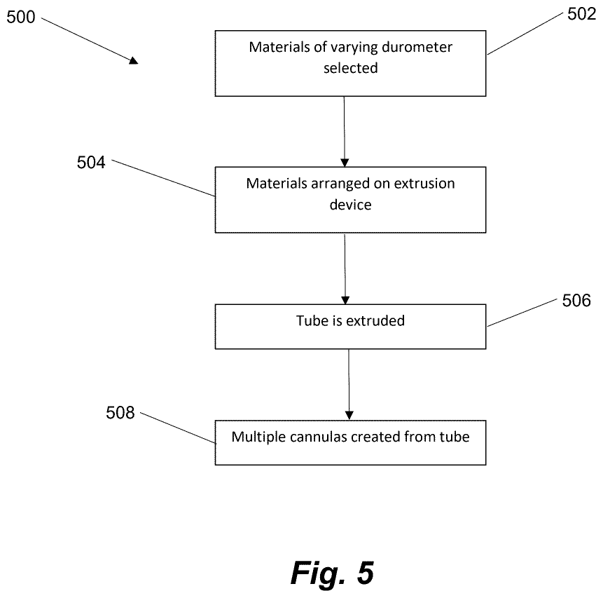

Referring now to FIG. 5, a flowchart 500 of method steps for a method of manufacturing a cannula such as, for example, cannula 406, for use in an infusion set is depicted. At step 502, two different materials having varying durometers are selected for the soft outer tube and the rigid core of the cannula. These materials are arranged on the extrusion device at step 504. The extrusion process is carried out at step 506. This includes rotating the tube during the extrusion process to form the rigid material in a helical shape within the outer tube. Following extrusion, the tube can be processed into multiple cannulas at step 508. This can include, for example, cutting the tube to length, providing a tapered tip at the distal end and laser drilling infusion holes in the sides of the tube.

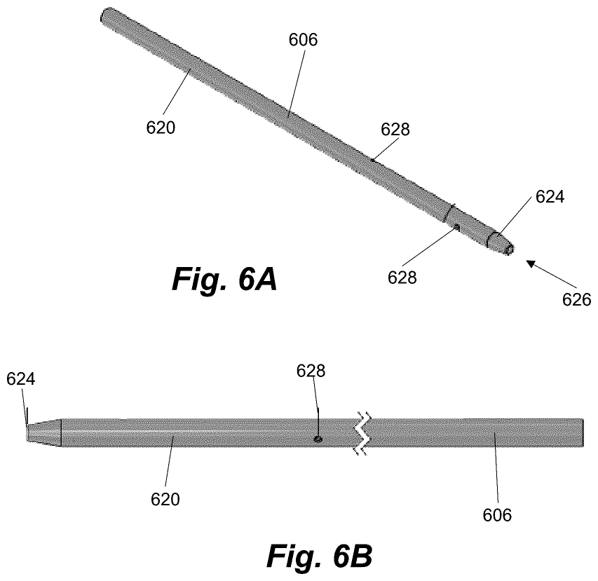

A cannula 606 formed from such a process is depicted in FIGS. 6A-6B. Cannula 606 comprises an elongate tube 620 having an inner lumen (not pictured). Cannula 606 further includes a tapered distal end 624 having a distal opening 626 and one or more infusion holes 428 along the tube wall. The elongate tube 620 can have rigid helical cores embedded therein that mimic the functionality of a cannula having a stainless-steel coil in the inner lumen as discussed above.

The cannula and cannula manufacturing process disclosed herein simplifies the upstream manufacturing process. There is a cost reduction in eliminating the stainless-steel coil and mandrel (which is otherwise discarded after manufacturing) while maintaining the functional advantages of flexibility and kink prevention of the cannula. Material handling is simplified by eliminating the need to carefully mount stainless steel coils on a mandrel, keeping the coil spacing consistent throughout the co-extrusion process and thereafter removing the mandrel. An additional advantage of the disclosed cannula is that by eliminating the use of a metal material, the cannula is corrosion resistant and safe to remain inserted during magnetic and CT scans.

In an embodiment, a cannula configured to facilitate delivery of insulin from an infusion pump to a user can include an elongate tube having an outer wall and an inner lumen. A helical core can be disposed within the outer wall and extend along a length of the elongate tube. The helical core can be comprised of a more rigid material than the elongate tube.

In embodiments, the helical core comprises a single, continuous core.

In embodiments, the helical core comprises a plurality of overlapping cores.

In embodiments, the entirely of the helical core is disposed within the outer wall.

In embodiments, the helical core is partially disposed within the outer wall such that a portion of the helical core is disposed within the inner lumen of the elongate tube.

In embodiments, the elongate tube comprises a tapered distal end and a distal opening.

In embodiments, the elongate tube comprises one or more infusion holes along the length of the elongate tube.

In embodiments, the elongate tube and the helical core are both formed from a polymer material.

In an embodiment, a cannula can be configured to facilitate delivery of insulin from an infusion pump to a user. The cannula can be formed with a helical core disposed within and extending along a length of an outer wall of an elongate tube. The helical core can be comprised of a more rigid material than the elongate tube.

In embodiments, the cannula is formed by an extrusion process.

In embodiments, the helical core is formed within the outer wall by rotating the elongate tube during the extrusion process.

In embodiments, the helical core and the elongate tube are both formed from a polymer material.

In an embodiment, an insulin delivery system includes a cannula including an elongate tube having an outer wall and an inner lumen and a helical core disposed within the outer wall and extending along a length of the elongate tube. The helical core can be comprised of a more rigid material than the elongate tube.

In embodiments, the system further includes an infusion pump configured to deliver insulin from the infusion pump to a user through the cannula.

In embodiments, the system further includes an infusion set incorporating the cannula and infusion tubing extending from the infusion pump to the infusion set.

In embodiments, the helical core comprises a single, continuous core.

In embodiments, the helical core comprises a plurality of overlapping cores.

In embodiments, the entirely of the helical core is disposed within the outer wall.

In embodiments, the helical core is partially disposed within the outer wall such that a portion of the helical core is disposed within the inner lumen of the elongate tube.

In embodiments, the elongate tube and the helical core are both formed from a polymer material.

Although the infusion pump embodiments herein are specifically described primarily with respect to the delivery of insulin, delivery of other medicaments, singly or in combination with one another or with insulin, including, for example, glucagon, pramlintide, etc., as well as other applications are also contemplated. Device and method embodiments discussed herein may be used for pain medication, chemotherapy, iron chelation, immunoglobulin treatment, dextrose or saline IV delivery, treatment of various conditions including, e.g., pulmonary hypertension, or any other suitable indication or application. Non-medical applications are also contemplated.

Also incorporated herein by reference in their entirety are commonly owned U.S. Pat. Nos. 6,999,854; 8,133,197; 8,287,495; 8,408,421 8,448,824; 8,573,027; 8,650,937; 8,986,523; 9,173,998; 9,180,242; 9,180,243; 9,238,100; 9,242,043; 9,335,910; 9,381,271; 9,421,329; 9,486,171; 9,486,571; 9,492,608; 9,503,526; 9,555,186; 9,565,718; 9,603,995; 9,669,160; 9,715,327; 9,737,656; 9,750,871; 9,867,937; 9,867,953; 9,940,441; 9,993,595; 10,016,561; 10,201,656; 10,279,105; 10,279,106; 10,279,107; 10,357,603; 10,357,606; 10,492,141; 10/541,987; 10,569,016; 10,736,037; 10,888,655; 10,994,077; 11,116,901; 11,224,693; 11,291,763; 11,305,057; 11,458,246; 11,464,908; 11,654,236; 11,911,595; 12,138,425; and 12,214,159 and commonly owned U.S. Patent Publication Nos. 2009/0287180; 2012/0123230; 2013/0053816; 2014/0276423; 2014/0276569; 2014/0276570; 2018/0071454; 2019/0307952; 2020/0206420; 2020/0329433; 2020/0372995; 2021/0001044; 2021/0113766; 2022/0062553; 2022/0139522; 2022/0223250; 2022/0233772; 2022/0233773; 2022/0238201; 2022/0265927; 2023/0034408; 2022/0344017; 2022/0370708; 2022/0037465; 2023/0040677; 2023/0047034; 2023/0113545; 2023/0113755; 2023/0166033; 2023/0166037; 2023/0173170; 2023/0201452; 2023/0241314; 2023/0277765; 2023/0338653; 2023/0381406; 2024/0050650; 2024/0226423; 2024/0226424 and 2024/0277924; 2024/0399051; 2024/408303; 2024/0416032; 2024/0416033; 2025/0099674; 2025/0099675 2025/0099678; 2025/0099679; and 2025/0108162 and commonly owned U.S. patent application Ser. Nos. 17/368,968; 17/896,492; 18/398,543; 18/962,169; 19/003,140; 19/003,164 and 19/119,554.

With regard to the above detailed description, like reference numerals used therein may refer to like elements that may have the same or similar dimensions, materials, and configurations. While particular forms of embodiments have been illustrated and described, it will be apparent that various modifications can be made without departing from the spirit and scope of the embodiments herein. Accordingly, it is not intended that the invention be limited by the forgoing detailed description.

The entirety of each patent, patent application, publication, and document referenced herein is hereby incorporated by reference. Citation of the above patents, patent applications, publications and documents is not an admission that any of the foregoing is pertinent prior art, nor does it constitute any admission as to the contents or date of these documents.

Modifications may be made to the foregoing embodiments without departing from the basic aspects of the technology. Although the technology may have been described in substantial detail with reference to one or more specific embodiments, changes may be made to the embodiments specifically disclosed in this application, yet these modifications and improvements are within the scope and spirit of the technology. The technology illustratively described herein may suitably be practiced in the absence of any element(s) not specifically disclosed herein. The terms and expressions which have been employed are used as terms of description and not of limitation and use of such terms and expressions do not exclude any equivalents of the features shown and described or portions thereof and various modifications are possible within the scope of the technology claimed. Although the present technology has been specifically disclosed by representative embodiments and optional features, modification and variation of the concepts herein disclosed may be made, and such modifications and variations may be considered within the scope of this technology.

1. A cannula configured to facilitate delivery of insulin from an infusion pump to a user, comprising:
an elongate tube having an outer wall and an inner lumen;
a helical core disposed within the outer wall and extending along a length of the elongate tube,
wherein the helical core is comprised of a more rigid material than the elongate tube.

2. The cannula of claim 1, wherein the helical core comprises a single, continuous core.

3. The cannula of claim 1, wherein the helical core comprises a plurality of overlapping cores.

4. The cannula of claim 1, wherein the entirely of the helical core is disposed within the outer wall.

5. The cannula of claim 1, wherein the helical core is partially disposed within the outer wall such that a portion of the helical core is disposed within the inner lumen of the elongate tube.

6. The cannula of claim 1, wherein the elongate tube comprises a tapered distal end and a distal opening.

7. The cannula of claim 1, wherein the elongate tube comprises one or more infusion holes along the length of the elongate tube.

8. The cannula of claim 1, wherein the elongate tube and the helical core are both formed from a polymer material.

9. A cannula configured to facilitate delivery of insulin from an infusion pump to a user, the cannula formed with a helical core disposed within and extending along a length of an outer wall of an elongate tube, wherein the helical core is comprised of a more rigid material than the elongate tube.

10. The cannula of claim 9, wherein the cannula is formed by an extrusion process.

11. The cannula of claim 9, wherein the helical core is formed within the outer wall by rotating the elongate tube during the extrusion process.

12. The cannula of claim 9, wherein the helical core and the elongate tube are both formed from a polymer material.

13. An insulin delivery system, comprising:
a cannula including an elongate tube having an outer wall and an inner lumen and a helical core disposed within the outer wall and extending along a length of the elongate tube,
wherein the helical core is comprised of a more rigid material than the elongate tube.

14. The insulin delivery system of claim 13, further comprising an infusion pump configured to deliver insulin from the infusion pump to a user through the cannula.

15. The insulin delivery system of claim 14, further comprising an infusion set incorporating the cannula and infusion tubing extending from the infusion pump to the infusion set.

16. The insulin delivery system of claim 13, wherein the helical core comprises a single, continuous core.

17. The insulin delivery system of claim 13, wherein the helical core comprises a plurality of overlapping cores.

18. The insulin delivery system of claim 13, wherein the entirely of the helical core is disposed within the outer wall.

19. The insulin delivery system of claim 13, wherein the helical core is partially disposed within the outer wall such that a portion of the helical core is disposed within the inner lumen of the elongate tube.

20. The insulin delivery system of claim 13, wherein the elongate tube and the helical core are both formed from a polymer material.
